(19) United States
(12) Patent Application Publication
(10) Pub. No.: US 2013/0317555 A1
(43) Pub. Date: Nov. 28, 2013

(54) IMPLANT AND SYSTEM FOR BONE REPAIR

(71) Applicant: Benvenue Medical, Inc., Santa Clara, CA (US)

(72) Inventors: Laurent Schaller, Lost Altos, CA (US); Sean Merrill Tutton, Shorewood, WI (US)

(73) Assignee: Benvenue Medical, Inc., Santa Clara, CA (US)

(21) Appl. No.: 13/897,862

(22) Filed: May 20, 2013

Related U.S. Application Data
(60) Provisional application No. 61/784,761, filed on Mar. 14, 2013.
(60) Provisional application No. 61/650,154, filed on May 22, 2012.

(51) Int. Cl. A61B 17/86; A61B 17/84

(52) U.S. Cl. CPC A61B 17/864; A61B 17/844
USPC 606/323; 606/300; 606/326

(57) ABSTRACT

Devices and methods for treating bones wherein the devices include a generally elongated implant that has a proximal end portion, a distal end portion and an intermediate portion therebetween. The implant also includes a lumen extending therethrough. The intermediate portion includes a configuration for engaging bone structure to substantially immobilize the implant within the bone structure. The intermediate portion also includes openings therein that are in communication with the lumen.

This application claims the benefit of U.S. Provisional Application No. 61/650,154, filed May 22, 2012, and U.S. Provisional Application No. 61/784,761, filed Mar. 14, 2013, both of which are hereby incorporated herein by reference.

### FIELD OF THE INVENTION

The present disclosure generally relates to apparatus and methods in the field of surgery and more particularly to apparatus and methods for use in minimally invasive orthopedic procedures for repairing bones, such as rib and clavicle bones.

### BACKGROUND OF THE INVENTION

Most bone fractures and other bone injuries are commonly treated with conventional fixation devices, such as splints, braces, casts and internal and external bone fixation implants, e.g., screws and plates. However, certain flat and long bones, such as the rib and clavicle bones, are rarely treated with conventional fixation devices because of the challenges presented by the size and location of such bones.

The treatments for injured rib and clavicle bones typically include rest along with application of a torso wrap for rib fractures and an arm sling for clavicle fractures. Treating rib and clavicle bone fractures in this manner oftentimes is very painful and an uncomfortable experience for the patient because such wraps and slings provide very little in the way of stability and immobilization to the injured bone. Additionally, the lack of stability and immobilization provided by such treatments increases the time it takes for the bones to heal relative to those bones that are treated with conventional fixation devices.

### SUMMARY

The devices and methods of the present disclosure may be used in minimally invasive surgical procedures to treat fractured or otherwise damaged bones, e.g., damaged by cancer or other disease. Such devices and methods may be particularly useful in the treatment of flat and long bones and, in particular, rib and clavicle bones. The devices and methods also may be used in the treatment of other types of bones as well. Additionally, the devices and methods also may be used in conjunction with other treatment devices and systems, such as bone screws and plates.

The devices of the present disclosure are inserted into a damaged bone to stabilize and immobilize the bone. Stabilizing the bone assists the healing process and can help reduce pain and healing time. Optionally, the devices also may create a platform for delivery and placement of therapeutic material, such as osteoconductive material, bone stabilizing materials, therapy specific drugs or combinations thereof.

In one aspect of the present disclosure, a device for treating bone is provided. The device includes a generally elongated implant that has a proximal end portion, a distal end portion and a flexible intermediate portion therebetween. The implant also includes a lumen extending therethrough. The implant includes a proximal or distal end opening in fluid communication with the lumen for receiving material into the lumen. The flexible intermediate portion has a configuration that engages bone structure to substantially immobilize the implant within the bone. In one embodiment, the configuration of the intermediate portion engages bone to provide pull-out resistance. The flexible intermediate portion also includes at least one opening in fluid communication with the lumen wherein material within the lumen exits through the opening of the intermediate portion.

In another aspect, a system for treating a bone is provided. The system includes a generally elongated guide member and a generally elongated implant configured to be guided by the guide member into the bone. The guide member includes a distal end portion configured for advancement into bone. The implant has a proximal end portion, a distal end portion and a flexible intermediate portion therebetween. The implant also includes a guide lumen extending therethrough for receiving the elongated guide member to guide the implant into bone. The flexible intermediate portion of the implant has a configuration that engages bone structure to substantially immobilize the implant within the bone. The flexible intermediate portion also includes at least one opening in fluid communication with the guide lumen.

Yet another aspect provides a method for treating bone. The method includes inserting a generally elongated guide member into a bone, such as in the intramedullary cavity or canal, and advancing a generally elongated implant along the guide member and into the bone. The elongated implant includes a proximal end portion, a distal end portion and an intermediate portion. The elongated implant also includes a lumen extending therethrough and the intermediate portion includes at least one opening in fluid communication with the lumen. Furthermore, the intermediated portion has a configuration that engages bone structure to substantially immobilize the implant relative to the bone. The method also includes introducing a material into the lumen wherein the material passes through the lumen and exits through the at least one opening of the intermediate portion.

These and other aspects are set forth in the following detailed description. In that respect, it should be noted that the present disclosure includes a number of different aspects which may have utility alone and/or in combination with other aspects. Accordingly, the above summary is not exhaustive identification of each such aspect that is now or may hereafter be claimed, but represents an overview of the present invention to assist in understanding the more detailed description that follows. The scope of the invention is as set forth in the claims now or hereafter filed.

### BRIEF DESCRIPTION OF THE FIGURES

In the course of this description, reference will be made to the accompanying drawings, wherein:

FIG. 1 is a schematic perspective view of a rib cage;

FIG. 2 is a partial perspective view of a fractured rib;

FIG. 3A is a perspective view of one embodiment of an implant of the present disclosure;

FIG. 3B is a perspective view of another embodiment of an implant of the present disclosure;

FIG. 4A is a cross-sectional view of the implant shown in FIG. 3A;

FIG. 4B is a cross-sectional view of the implant shown in FIG. 3B;

FIG. 5A is a side view of another embodiment of an implant of the present disclosure;

FIG. 5B is a perspective view of another embodiment of an implant of the present disclosure;

FIG. 5C is a perspective view of another embodiment of an implant of the present disclosure;

FIG. 6 is a perspective view of various access tools that may be used during the implantation of the implant of the present disclosure;

FIG. 7 is a perspective view of one embodiment of an implant deployment device that may be used to deploy an implant of the present disclosure;

FIG. 8 is a perspective view of a fractured rib having a portion of the rib cutaway and shown with an access device inserted into the rib;

FIG. 9 is a perspective view of the fractured rib of FIG. 8 having a portion of the rib cutaway and shown with a guidewire inserted into the rib after the access device has been removed;

FIG. 10 is a perspective view of the fractured rib of FIG. 9 having a portion of the rib cutaway and shown with a working cannula advanced over the guidewire and into the rib;

FIG. 11 is a perspective view of the fractured rib of FIG. 10 having a portion of the rib cutaway and shown with a guide member inserted through a lumen of the working cannula and into a first section of the rib on one side of the fracture;

FIG. 12 is a perspective view of the fractured rib of FIG. 11 having a portion of the rib cutaway and shown with the guide member advanced across the fracture and into the second section of the rib, bridging the two sections of the fractured rib;

FIG. 13 is a perspective view of the fractured rib of FIG. 12 having a portion cutaway and shown with an implant advanced along the guide member and into the rib;

FIG. 14 is a perspective view of the fractured rib of FIG. 13 having a portion cutaway and shown with a therapeutic injection device operably engaged with the an opening in the proximal end portion of the implant;

FIG. 15 is a perspective view of the fractured rib of FIG. 14 having a portion cutaway and which has been stabilized by the implant;

FIG. 16 is a perspective view of one embodiment of an implant deployment device of the present disclosure;

FIG. 17 is a perspective view of another embodiment of an implant deployment device of the present disclosure;

FIG. 18 is a perspective view showing the deployment device of FIG. 16 delivering an implant of the present disclosure into a fractured rib;

FIG. 19 is a side view of another embodiment of an implant of the present disclosure shown within a treatment site;

FIG. 20 is a side view of the implant of FIG. 19;

FIG. 21 is a cross-sectional view of the distal end portion of the implant of FIG. 19; and

FIG. 22 is a cross-sectional view of the proximal end portion of the implant of FIG. 19.

### DETAILED DESCRIPTION

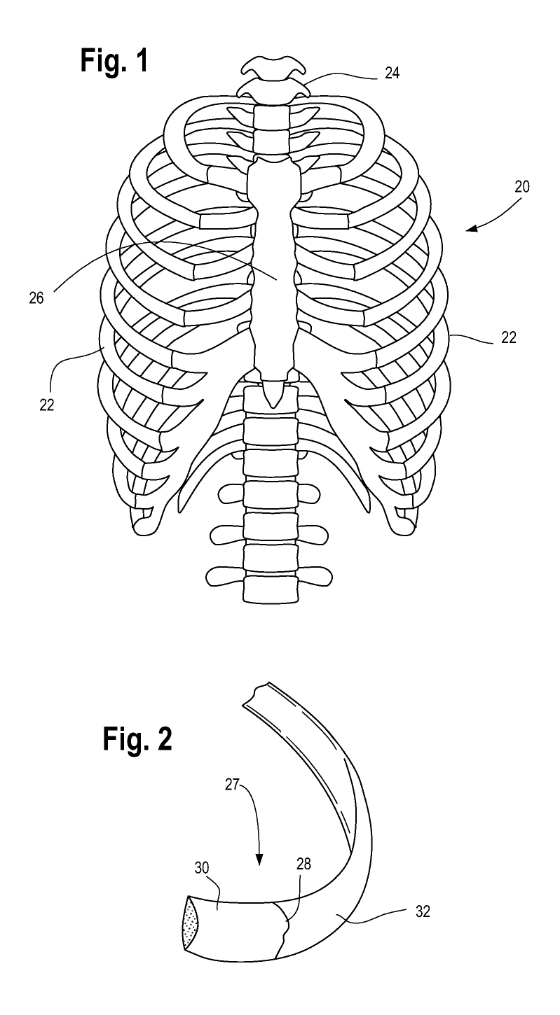

FIG. 1 is a schematic illustration of a rib cage 20 that includes ribs 22. Ribs 22 are connected to a vertebral column 24 and sternum 26. FIG. 2 illustrates a fractured rib 27 of rib cage 20 which includes a fracture 28 that divides rib 27 into two separate sections identified as 30 and 32. Because ribs are generally elongated flat bones that have relatively small cross-sectional dimensions, it can be difficult to treat and stabilize fractured ribs with traditional stabilization devices, such as casts, braces, plates and screws. Without stabilization, patients may experience sever pain and discomfort from motion between the fractured sections while the bone is healing. This also is often the occasion with other flat and long bones, such as the clavicle, humerus, tibia, phalanges, etc. It should be understood that while the devices and methods disclosed herein are done so relative to rib bones that the devices and methods are not limited to the treatment of rib bones and can be used to treat other bones and tissue as well.

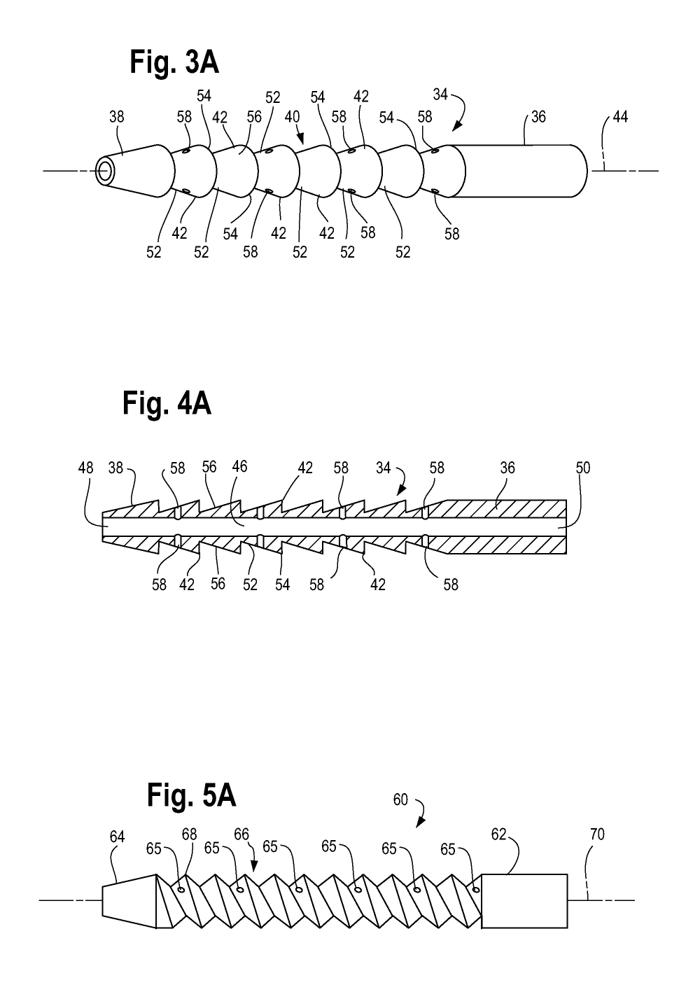

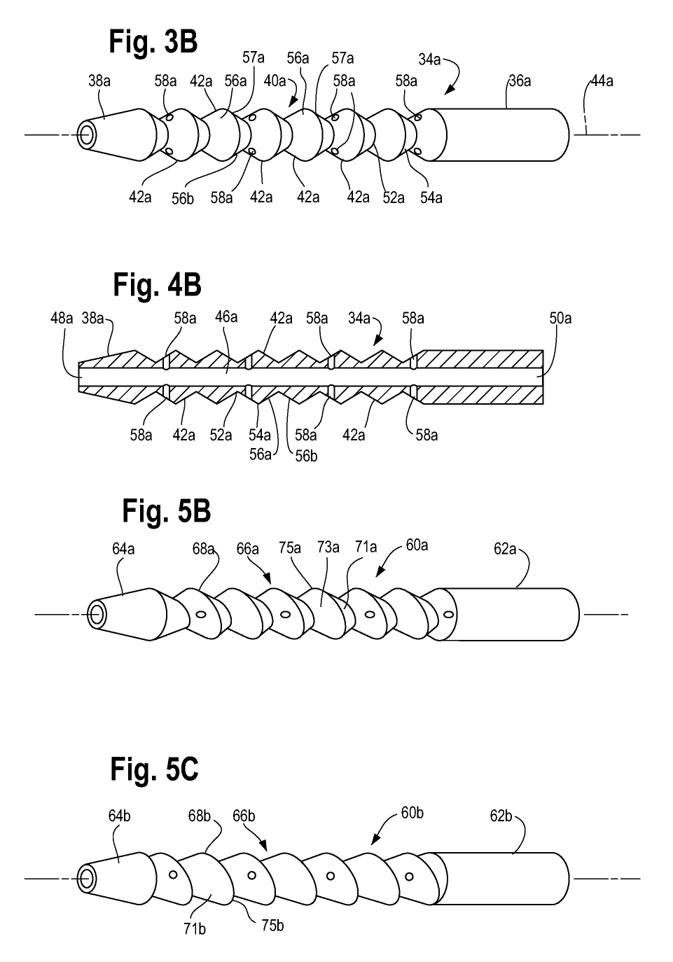

FIGS. 3A and 4A show one embodiment of an implant 34 of the present disclosure which may be implanted within a bone, such as a rib bone, for stabilization and immobilization thereof. Implant 34 is generally elongated and includes a proximal end portion 36, a distal end portion 38, and an intermediate portion 40 therebetween. As shown in FIG. 4A, elongated implant 34 includes a lumen 46 extending therethrough. Optionally, distal end portion 38 may include a distal end opening 48 and/or proximal end portion 36 may include a proximal end opening 50, both of which, when present, are in communication with lumen 46. As discussed in more detail below, proximal end opening 50, distal end opening 48 and lumen 46 may be configured to receive a guide member or receive therapeutic material therethrough.

The implant 34 and the other implants of the present disclosure may be made of any suitable biocompatible material, such as biocompatible polymers or metals. The polymers may be absorbable, non-absorbable or combinations thereof. When an absorbable polymer is used, it is preferable that the polymer be able to substantially maintain the structure of the implant and not lose substantial strength or be absorbed by the body until the bone has been sufficiently stabilized by the healing process or has been substantially healed. The implant may be made from polymers, such as ultrahigh molecular weight polyethylene, polyetheretherketone (PEEK), hydrogels or the like. The implants also may be made of one or more biocompatible metals or alloys such as titanium, steel, nickel-titanium alloys or the like. In other embodiments, the implants may be made from compressed bone growth or argumentation material that has been compressed or otherwise conditioned to form the shape of the implant. The implants may also be made of any combination of the above-described materials.

Turning back to FIG. 3A, intermediate portion 40 has a configuration that allows for flexibility and is preferably semi-flexible. More specifically, intermediate portion 40 preferably has enough flexibility to allow at least some bending or curving along a path while having sufficient rigidity and material strength to resist lateral shifting or movement and to provide stability to the damaged bone. The length of intermediate portion 40 and location of it between the proximal and distal tips of the implant may vary. As shown in FIG. 3A, for example, intermediate portion 40 may extend substantially to the distal end tip of the implant 34. In another embodiment, the intermediate portion 40 may extend substantially to the proximal end tip of the implant. In yet another embodiment, the implant also may be flexible along the entire length of the implant wherein the flexible section extends all the way to the proximal and distal end tips. Additionally, the flexible sections of intermediate portion 40 may be interrupted by non-flexible sections.

Intermediate portion 40 also may be configured to engage bone structure after implant 34 has been implanted within the bone. Such engagement assists in immobilizing implant 34 relative to the bone and limiting motion between the different segments of the fractured bone structure and stabilizing the same. In one embodiment implant 34 acts to retain, hold or locks the fractured bone pieces in place relative to one another. Intermediate portion 40's configuration and engagement with bone structure also may provide pullout resistance, which assists in maintaining implant 34 in place within the bone and prevents the implant from being pulled out of the bone, and more specifically pulled out of a section of fractured bone that the implant is stabilizing or bridging relative to an adjacent section of fractured bone.

In the embodiment shown in the FIGS. 3A and 4A, intermediate portion 40 includes a plurality of interconnected segments 42 spaced along the elongated axis 44 of implant 34. Segments 42 may be similarly configured in shape and size or the configurations between the segments may vary. Also, segments 42 may be integral (a single unitary structure) or may be a plurality of separate segments that are connected together. In the embodiment shown in FIGS. 3A and 4A, segments 42 are similarly shaped and sized and have a generally frustoconical shape in which the cross-sectional shape of the distal end portion 52 of segment 42 is smaller than the cross-sectional shape of the proximal end portion 54 of the segment. Thus, the outer surfaces 56 of each segment 42 are tapered toward distal end portion 52 of the segment 42 and proximal end portion 54 flares or inclines radially outwardly. Tapered surface 56 allows implant 34 to be inserted or pushed into the bone in a first distal direction along longitudinal axis 44 wherein the smaller cross-sectional distal end portion 52 of each segment 42 leads or enters the bone first. Once inserted, the larger cross-sectional proximal end portions 54 of segments 42 engage bone structure like a hook or barb to assist in immobilizing implant 34 within the bone, stabilizing the fractured bone sections relative to each other, and preventing implant 34 from being pulled out of the bone or fractured bone sections. In one embodiment, proximal end portions 54 of segments 42 may be resiliently flexible such that proximal end portions 54 flex or compress inward as each section 42 passes through bone material and then substantially returns to the initial shape once implanted so as to engage bone structure and resist withdrawal or pull-out.

Intermediate portion 40 may also include lateral or side openings 58 spaced apart along longitudinal axis 44 of implant 34 and spaced apart along the circumference of implant 34. As shown in FIG. 4, openings 58 are in fluid communication with lumen 46 such that material, for example therapeutic material, may flow through the lumen and out of openings 58.

FIGS. 3B and 4B illustrate another embodiment of an implant 34a of the present disclosure. Similar to implant 34, implant 34a includes a proximal end portion 36a, a distal end portion 38a and an intermediate portion 40a. Implant 34a may also, but not necessarily, include a proximal end opening 50a, a distal end opening 48a and a lumen 46a extending therebetween.

Intermediate portion 40a includes a plurality of interconnected segments 42a spaced along the elongated axis 44a of implant 34a. Segments 42a also may be similarly configured in shape and size or the configurations between the segments may vary. For example, intermediate portion 40a may include a mixture of segments wherein the segments include configurations of both segments 42a and segments 42. Additionally, segments 42a may be integral (a single unitary structure) or may be a plurality of separate segments that are connected together.

In the embodiment shown in FIGS. 3B and 4B, segments 42a are similarly shaped and sized. The segments 42a generally have the shape of two conical frustums abutted together at their bases, wherein the cones project in opposite directions, i.e., proximal and distal directions. The segments 42a include surfaces 56a and 56b which taper radially inwardly in opposite directions away from a substantially central region 57a of the segment 42a. Additionally, the cross-sectional shape of the central region 57a is larger than the cross-sectional shape of the proximal and distal ends 52a, 54a of the segments 42a.

Intermediate portion 40a may also include lateral or side openings 58a spaced apart along longitudinal axis 44a of implant 34a and spaced apart along the circumference of implant 34a. As shown in FIG. 4B, openings 58a are in fluid communication with a lumen 46a such that material, for example therapeutic material, may flow through the lumen and out of openings 58.

FIG. 5A illustrates another embodiment of an implant 60 of the present disclosure. In this embodiment, implant 60 includes a proximal end portion 62, a distal end portion 64, and an intermediate portion 66 therebetween. Implant 60 may optionally include a lumen (not shown) extending therethrough and, optionally, include openings 65 spaced along the intermediate portion in communication with the lumen. Intermediate portion 66 is threaded and includes one or more screw-like threads 68 extending helically around intermediate portion 66. To insert implant 60 into bone, the implant is rotated about longitudinal axis 70 to thread or screw implant 60 into the bone. As implant 60 is rotated, threads 68 engage bone structure and such engagement assists in advancing implant 60 into the bone, reducing the need for axial impact or hammered insertion.

FIGS. 5B and 5C illustrate further embodiments of implants 60a and 60b of the present disclosure. Implants 60a and 60b are similar to implant 60 in that each includes a proximal end portion 62a/62b, a distal end portion 64a/64b and an intermediate portion 66a/66b. Additionally, implants 60a and 60b may also optionally include a lumen extending between proximal and distal end openings and, optionally, include openings spaced along the intermediate portion and in communication with the lumen.

In the implant 60a of FIG. 5B, the intermediate portion 66a includes a thread 68a wherein the thread 68a includes surfaces 71a/73a on opposite sides of the thread crest 75a. Surface 71a tapers radially inwardly in the proximal direction and surface 73a tapers radially inwardly in the distal direction away from crest 75a. In the implant of FIG. 5C, intermediate portion 66b has a thread 68b that includes a surface 71b that extends distally of thread crest 75b. The surface 71b tapers radially inwardly in the distal direction from the thread crest 75b to the thread root.

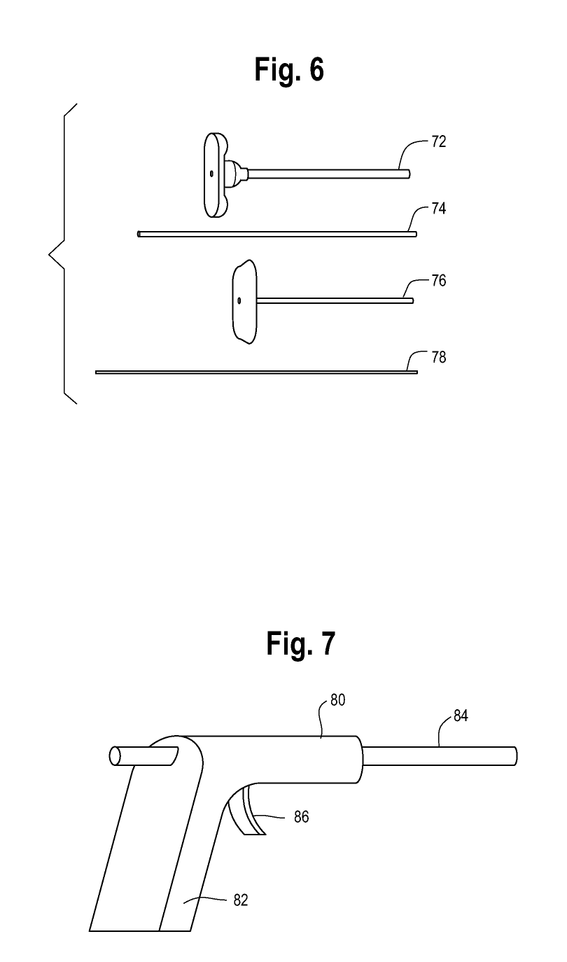

FIG. 6 illustrates various access tools that may be used to obtain access and deploy an implant of the present disclosure into the bone. Such access tools may include an initial access device 72, such as a Jamshidi needle, that may be used to initially pierce through the patient's skin, bone and other tissue. The tools also may include a guidewire 74, e.g. a K-wire, for guiding access instruments and other devices through the access site and into bone. The tools shown herein also include a working or access cannula 76 which may be used as the access cannula through which the implant is deployed into the bone. Working cannula 76 may also be used as a dilator to dilate and expand the access path into the bone. Finally, the tools may include a hollow guide member or cannula 78 for guiding an implant into the bone.

FIG. 7 illustrates one embodiment of an implant deployment device 80. Deployment device 80 includes a handle 82 and a cannula 84 extending therefrom and for passing the implant therethrough. Device 80 also includes an actuator 86 for advancing the implant through cannula 84. Deployment device 80 may include a ratchet type deployment system, a rotatable mechanism, both or other mechanism for advancing the implant.

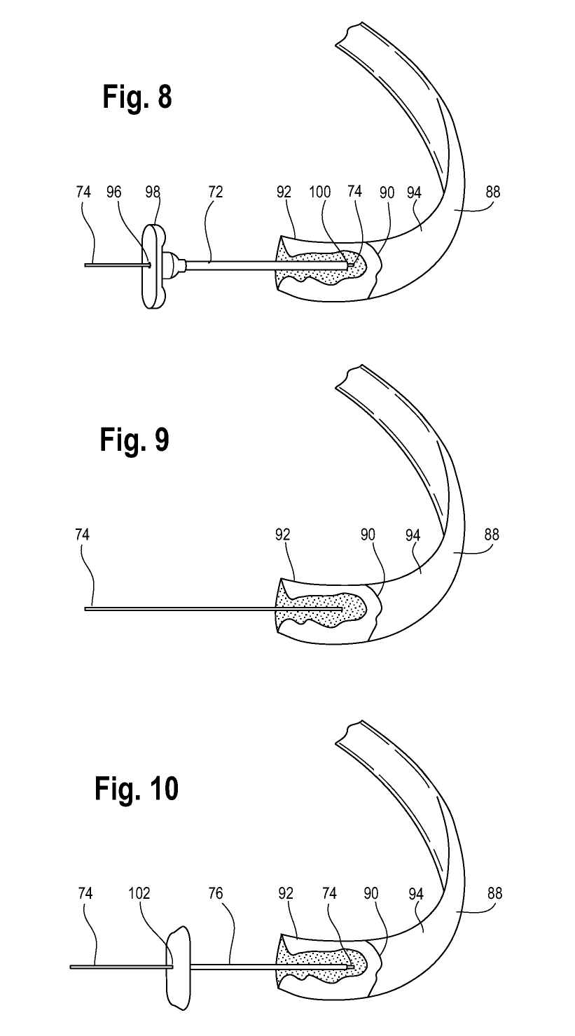

FIGS. 8-15 illustrate the deployment of an implant into a bone. Referring to FIG. 8, there is shown fractured bone 88 including a fracture 90 between sections 92 and 94 of the bone. The device may be used for other and multiple fractures also. Initial access device 72 pierces the outer bone wall of fractured bone 88 to obtain access to the interior of the bone. Initial access device 72 may be percutaneously inserted through the patient's skin and other tissue to access bone 88 in a minimally invasive procedure. Alternatively, initial access device 72 may be inserted into bone 88 during a traditional open surgery. After initial access device 72 is inserted to the desired position, such as in the intramedullary canal, guidewire 74 (e.g., a K-wire) is inserted through an opening 96 in the proximal end portion 98 of initial access device 72. Guidewire 74 is advanced through a lumen (not shown) of initial access device 72 and extends out an opening in the distal end portion 100 of initial access device 72. After guidewire 74 has been inserted to the desired position, the initial access device 72 is removed from the surgical site, leaving the guidewire 74 in place, as shown in FIG. 9.

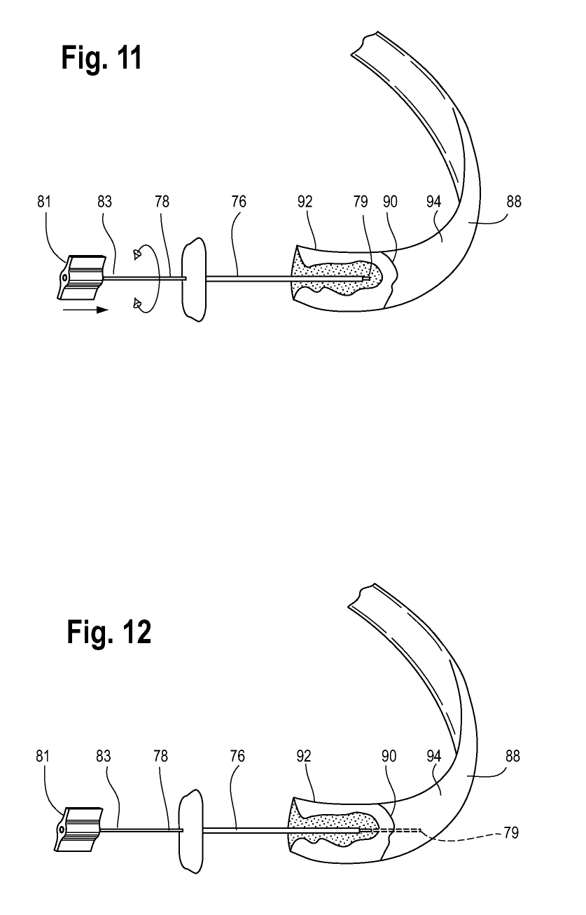

Referring to FIG. 10, guidewire 74 is passed through a lumen 102 of working cannula 76 and working cannula 76 is advanced over the guidewire 74 until working cannula 76 is at a desired position relative to bone 88. The cannula inserted over the guide wire may act to dilate the access opening into the bone. As illustrated in FIG. 11, guidewire 74 is retracted from the surgical site through working cannula 76. The generally elongated guide member 78 is advanced through working cannula 76 until, in the illustrated embodiment, a distal end 79 of guide member 78 is located in first section 92 of bone 88. While advancing guide member 78, the guide member may be rotated about the longitudinal axis which assists in advancing guide member 78 into bone 88. Additionally, guide member 78 may include a removable hub 81 located at proximal end 83 of guide member 78 that allows a user to grasp the guide member for manipulation of the guide member 78.

Turning now to FIG. 12, guide member 78 is further advanced into bone 88, across fracture 90 so that distal end portion 79 of guide member 78 extends into second section 94 of fractured bone 88. Thus, guide member 78 spans or bridges the fracture

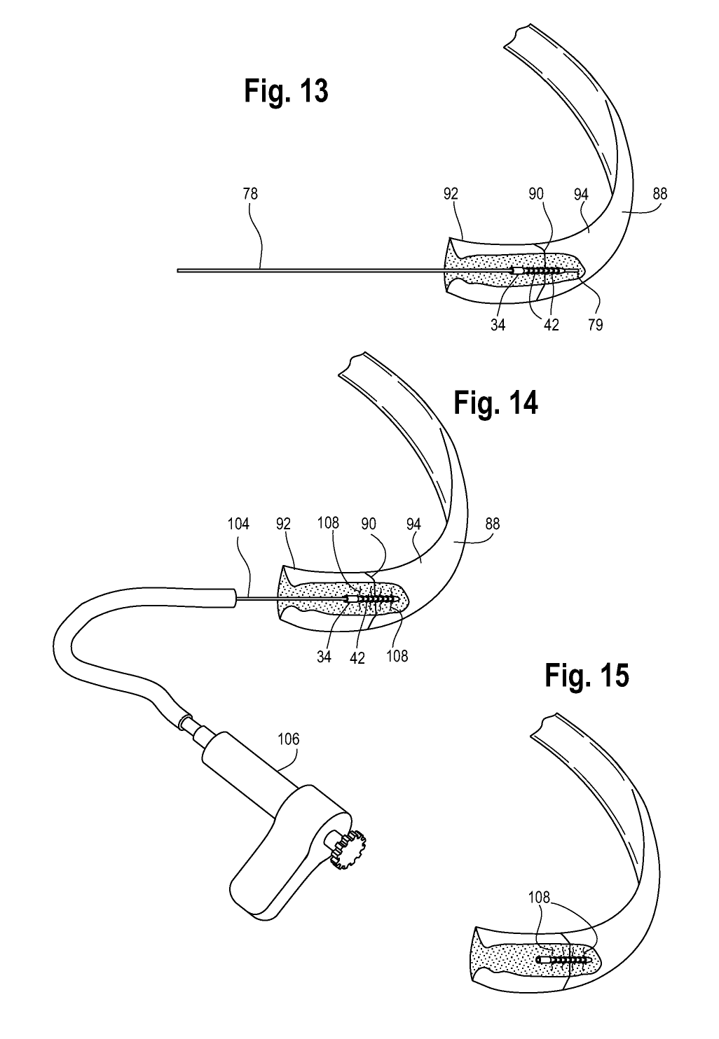

Hub 81, when present, is removed and guide member 78 is inserted through the lumen 46 (FIG. 4) of implant 34, and implant 34 is advanced over guide member 78 into bone 88 until implant 34 reaches the desired position within bone 88, as shown in FIG. 13. The advancement of implant 34 along guide member 78 may take place through working cannula 76 or after working cannula 76 has been removed or retracted. In the embodiment illustrated in FIG. 13, working cannula 76 has been removed. The implant 34 in this embodiment is advanced into section 92, across fracture 90 and into bone section 94. Thus, implant 34 is implanted in both sections 92 and 94 of bone 88 and spans fracture 90. If necessary, the physician may adjust or reposition the bone segments, to move them into alignment before insertion of the implant.

Segments 42 engage the bone structure of bone 88 to secure implant 88 in place within bone 88 and to substantially immobilize implant 34 within bone 88. Implant 34 also limits or reduces movement between the two sections 92 and 94 of bone 88 laterally and axially (along the axis of the implant). Turning now to FIG. 14, after implant 34 is implanted in the desired position within bone 88, guide member 78 is removed and, if desired, an injection cannula 104 of an injection device 106 may be operably connected to proximal end opening 50 (shown in FIG. 4) of implant 34. Injection device 106 injects flowable material 108 into lumen 46 (shown in FIG. 4) of implant 34. Flowable material 108 advances through lumen 46 and out of openings 58 located in intermediate portion 40 of implant 34 (shown in FIGS. 3 and 4). After the desired amount of flowable material 108 has been deployed, injection device 106 is retracted. The flowable material may be osteoconductive material, bone stabilizing materials, therapy specific drugs or combinations thereof. In one embodiment the therapeutic material is bone cement, such as PMMA. In another embodiment, the therapeutic material is an osteoconductive material that promotes bone growth. FIG. 15 illustrates a bone stabilized by implant 34.

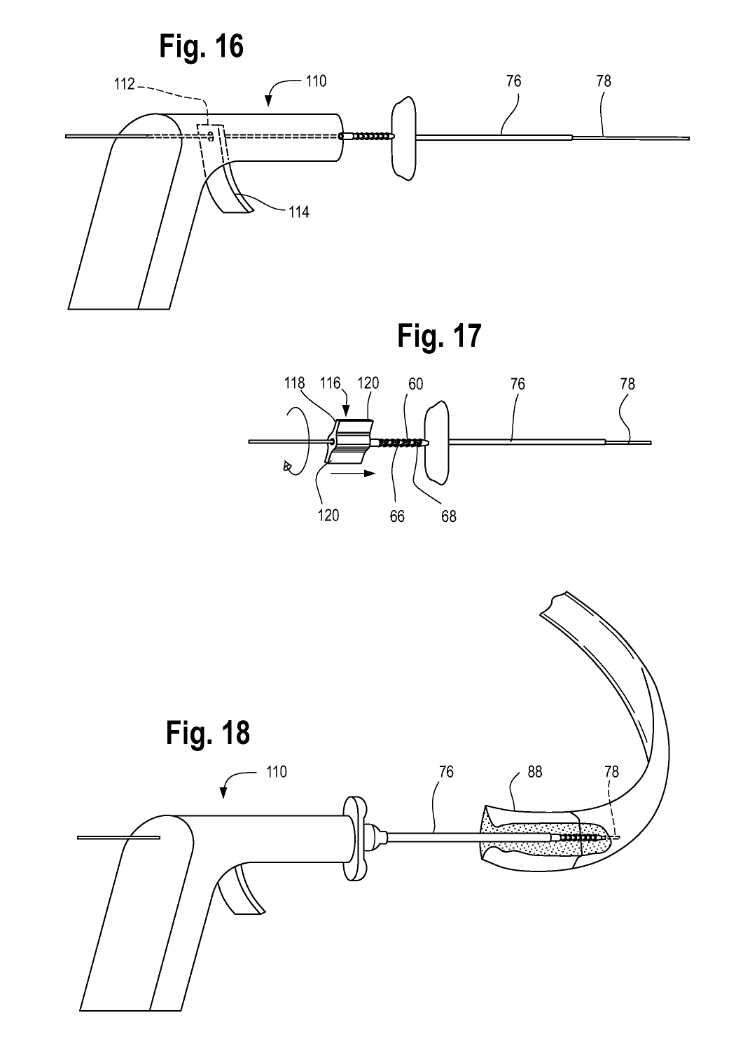

FIGS. 16 and 18 illustrate one embodiment of an implant deployment device 110 for deploying implant 34 through working cannula 76 and along guide member 78. Deployment device 110 includes a ratchet mechanism 112 for advancing implant 34 along guide member 78 and an actuator 114 for activating ratchet mechanism 112. As shown in FIG. 18, deployment device 110 may be used to advance implant 34 through working cannula 78, along guide member 76 and into bone 88.

FIG. 17 illustrates another embodiment of a deployment device 116 for delivering the threaded implant 60 into bone. Deployment device 116 includes a rotatable body 118 that releasably engages proximal end 62 of implant 60 in a manner such that rotation of body 118 rotates implant 60. In the illustrated embodiment, a pair of opposed wings 120 project radially outwardly from the rotatable body 118. In this embodiment, the user or an automated rotation device contacts wings 120 to rotate body 118. Implant 60 may be advanced through working cannula 76 and along guide member 78 into bone. Once intermediate portion 66 of implant 60 is in contact with bone, rotatable body 118 is rotated to rotate implant 60. As implant 60 is rotated threads 68 on intermediate potion 66 engage bone and implant 60 is screwed into the bone. Threads 68 assist in immobilizing implant 60 within the bone and limiting or reducing movement the pieces of fractured bond.

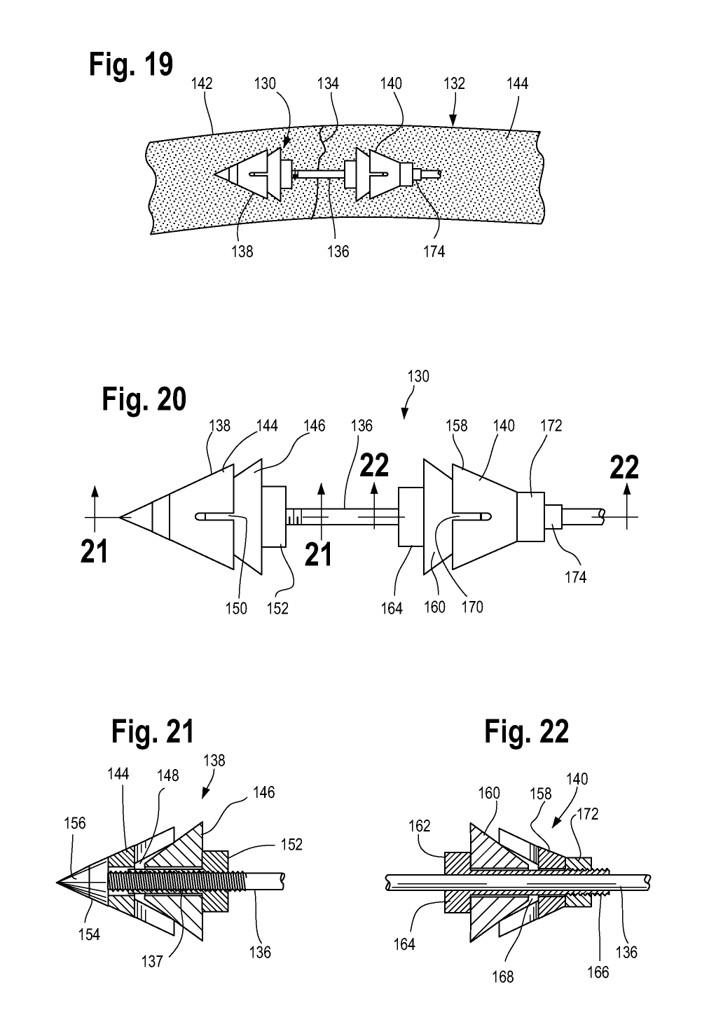

FIGS. 19-22 illustrate another implant 130 of the present disclosure. FIG. 19 shows the generally elongated implant 130 implanted within a bone 132, such as a rib or clavicle bone, and spanning or bridging fracture 134. Implant 130 includes an elongated member, such as a shaft or rod 136. A distal locking, retaining or anchor member 138 is located at or near the distal end portion of the rod 136 and a proximal locking, retaining or anchor member 140 is located at or near the proximal end portion of the rod 136. The anchor members 138, 140 engage bone tissue to anchor or retain the anchor members 138, 140 therein. Once anchored within the bone, anchor members 138, 140 may be moveable toward each other along rod 136 to pull the fractured sections 142, 144 together and hold the sections in a tight or compressed engagement. The anchor members 138, 140 may be moved toward each other by moving either one or both of anchor members 138, 140 toward the other anchor member along rod 136. Once the anchor members 138, 140 are in a desired position, one or both of the anchor members may be locked into position.

Referring to FIGS. 20-22, the proximal and distal anchor members 138, 140 may be expandable anchor members that are deployed into the bone in a first unexpanded configuration and then are expanded within the bone to anchor or secure the anchor members 138, 140 into the bone. The anchor members 138, 140 may include surfaces, barbs or hooks that anchor or secure the anchor members 138, 140 in the bone when the anchor member 138, 140 are in the expanded configuration.

Turning to FIGS. 20 and 21, distal anchor member 138 may include an expandable portion 144 and an expander member 146. As illustrated in FIG. 21, expandable portion 144 may have a generally frusto-conical shape including a lumen therethrough which has the distal end portion 137 of the rod 136 disposed therein. The expandable portion 144 defines a cavity or hollow 148 that receives expander member 146. Referring to FIG. 20, the expandable portion 144 may include slots 150 that accommodate radial outward expansion of the expandable portion 144. Expander member 146 may also have a generally frusto-conical shape and a lumen therethrough having the distal end potion 137 of rod 136 disposed therein. The anchor member 138 also includes a locking or tightening member 152 that may be used to advance the expander member 146 into the cavity 148 of the expandable portion 144 to expand the expandable portion 144. As the expander member 146 advances distally, the tapered surface of the expander member 146 contacts the inner surface of the expandable portion 144 to flare the expandable portion 144 radially outwardly to contact and anchor or secure the anchor member 138 into the bone. The implant 130 may include a stop 154 at the distal end thereof to hold or retain the expandable portion 144 in place while the expander member 146 is advanced distally into cavity 148 of the expandable portion 144. In one embodiment, the stop 154 may be the distal tip 156 of the shaft or the rod 136, which preferably, but not necessarily, is a pointed or sharp tip. The tightening member 152 may be a nut wherein the rod 136 includes threads such that as the nut is rotated it moves distally to advance expander member 144. Alternatively, the tightening member may be a cinching member that is incrementally pushed over barbs or ribs on the surface of the rod 136 and is locked into place.

Referring to FIGS. 20 and 22, proximal anchor member 140 may include an expandable portion 158, an expander member 160 and an expanding mechanism 162, such as a sliding or pushing mechanism, for sliding or pushing the expandable portion 158 onto the expander member 160 to expand the proximal anchor member 140. As illustrated in FIG. 22, the expanding mechanism 162 includes a stop member 164 located distally of the expander member 160 wherein the stop member 164 holds the expander member 160 in place as the expandable portion 158 is pressed onto the expander member 160. The stop member 164 may be a plate abutting the distal end portion of the expander member 160 or a structure attached to the expander member 160. The expanding mechanism 162 also includes a shaft 166 having a lumen therethrough for receiving rod 136.

The expandable portion 158 may have a generally frusto-conical shape including a lumen for receiving the shaft 166 of the expanding mechanism. The expandable portion 158 defines a cavity or hollow 168 that receives expander member 160. Referring to FIG. 20, the expandable portion 158 may include slots 170 that accommodate radially expansion of the expandable portion 158. Expander member 160 may also have a generally frusto-conical shape and a lumen therethrough for receiving shaft 166 of the expanding mechanism 162. The proximal anchor 140 also include a locking or tightening member 172 associated with a proximal end portion of shaft 166 of the expanding mechanism wherein the proximal end portion 166 extends proximally beyond the expandable portion 158. The tightening member 172 may be used to advance the expandable portion 158 toward the expander member 160 such that expander member 160 enters into the cavity 168 of the expandable portion 158 to expand the expandable portion 158. As the expandable portion 158 advances distally, the tapered surface of the expander member 160 contacts the inner surface of the expandable portion 158 to flare the expandable portion 158 radially outward to contact bone and anchor or secure the proximal anchor member 140 into the bone. The tightening member 172 may be a nut wherein the shaft 166 of the expanding mechanism 162 includes threads such that as the nut is rotated it moves distally to advance expandable portion 158. Alternatively, the tightening member 172 may be a cinching member that is incrementally pushed over barbs or ribs on the surface of the shaft 166 and is locked into place.

Referring to FIGS. 19 and 20, implant 130 also includes a pusher, locking or tightening member 174 that is employed to advance the proximal anchor member 140 toward the distal anchor member 138. After the proximal and distal anchor members 138, 140 have been expanded, the pusher 174 is employed to push the proximal anchor member 140 toward the distal anchor member 138 to pull and hold fraction section 142, 144 together. The pusher 174 may include a nut wherein the rod 136 is threaded such that the nut advances distally as it is turned. In another embodiment, the pusher may include a cinching member that is incrementally pushed over barbs or ribs on the surface of the rod 136 and locked into place as it passes over the barbs or ribs.

In one method of employing implant 130, rod 136, preferably having the distal anchor member 138 disposed thereon and in an unexpanded configuration, is inserted into the bone 132 and across the fracture 134. In one embodiment, the rod 136 may have a lumen therethrough for receiving a guide member, such as a guide wire, such that the rod 136 may be advanceable over the guide member into the bone 132 and across the fracture 134. After the rod 136 and distal anchor member 138 are in the desired position, the distal anchor member 138 is expanded so as to anchor or secure the distal anchor member 138 into the bone tissue. When the tightening member 152 of distal anchor member 138 is a threaded nut, a cannulated wrench having a lumen for receiving the rod 136 may be inserted over the rod 136 to engage the nut. The cannulated wrench may be employed to rotate the nut to move the expander member 146 into cavity 148 of the expandable portion 144 of distal anchor member 138. When the tightening member is a cinch nut, a pusher, such as a cannulated pusher that is insertable over the rod 136 may be used to advance the cinch nut distally.

After the distal anchor member 138 has been expanded a desired amount, the proximal anchor member 140 in an unexpanded configuration is advanced over the rod 136 and positioned at a desired location within the bone 132. After the proximal anchor member 140 is in the desired location, it is expanded to anchor or secure the proximal anchor member 140 within the bone. For example, the tightening member 172 of anchor member 140 is advanced distally to move expandable portion 158 over expander member 160 to expand the expandable portion 158. When the tightening member 172 is a nut, a wrench such as a cannulated wrench, may be used to rotate the tightening member. When the tightening member 172 is a cinch member, a pusher may be used to advance the tightening member.

Once the proximal and distal anchor members 140, 138 have been expanded, the anchor members 138, 140 are moved toward each other to move and hold the fractured sections 142, 144 together. In the illustrated embodiment, proximal anchor member 140 is move toward distal anchor member 138 by pusher 174, which may be a nut or a cinching member.

Although the present invention is described in light of the illustrated embodiments, it is understood that this for the purposes illustration and not limitation. Other applications, modifications or use of the support or distraction device may be made without departing for the scope of this invention, as set forth in the claims now or hereafter filed.

1. A device for treating a bone, comprising:
a generally elongated implant having a proximal end portion, a distal end portion and a flexible intermediate portion therebetween, the implant including a lumen extending through the elongated implant, the implant also including a proximal or a distal end opening which is in fluid communication with the lumen and for receiving material therethrough and into the lumen; and
the flexible intermediate portion having a configuration that engages bone structure to resist movement of the elongated implant within the bone, and the flexible intermediate portion including at least one opening in fluid communication with the lumen wherein material within the lumen exits through the opening of the intermediate portion.

2. The device of claim 1 in which the configuration of the flexible intermediate portion comprises a plurality of retaining surfaces spaced axially there along or a threaded configuration.

3. The device of claim 1 in which the configuration of the flexible intermediate portion comprises a plurality of segments spaced along a longitudinal axis of the implant.

4. The device of claim 3 in which at least one segment has a proximal end portion and a distal end portion and the distal end portion of the segment has a smaller cross-sectional size than the proximal end portion.

5. The device of claim 3 in which at least one segment has an outer surface that is tapered toward the distal end portion of the elongated implant.

6. The device of claim 3 in which at least one segment has a generally frusto-conical shape.

7. The device of claim 1 in which the lumen is adapted to receive a guide member therethrough.

8. The device of claim 1 in which the proximal or distal end opening is adapted to operably engage a material injection device.

9.-20. (canceled)

21. An implant for treating bone comprising:
an elongated member including proximal and distal anchor members, wherein the proximal and distal anchor members are configured for anchoring into sections of fractured bone, and wherein either one or both of the anchor members is moveable toward the other anchor member to move the bone section together.

22. The implant of claim 21 wherein the proximal and distal anchor members are located on a rod.

23. The implant of any of claim 21 wherein at least one of the proximal and distal anchor members is expandable.

24. The implant of claim 23 wherein the anchor members include an expandable portion and an expander member configured to expand the expandable portion.

25. The implant of claim 24 wherein the expandable portion includes a generally frusto-conical shape.

26. The implant of any of claim 21 wherein the proximal anchor member is moveable toward the distal anchor member.

27. The implant of claim 26 further including a pusher for advancing the proximal anchor member toward the distal anchor member.
